(19) United States
(12) Patent Application Publication
(10) Pub. No.: US 2002/0086868 A1
(43) Pub. Date: Jul. 4, 2002

(54) Methods of use of gemifloxacin compounds against fluoroquinolone resistant Streptococcus pneumoniae bacteria

(76) Inventors: Darren Bast, Hamilton (CA); Ross J. Davidson, Nova Scotia (CA); Joyce DeAzavedo, Toronto (CA); Donald E. Low, Toronto (CA)

Correspondence Address: GLAXOSMITHKLINE, Corporate Intellectual Property - UW2220, P.O. Box 1539, King of Prussia, PA, 19406-0939, US

(21) Appl. No.: 10/041,963

(22) Filed: Dec. 19, 2001

Related U.S. Application Data
(63) Continuation of application No. 09/577,731, filed on May 23, 2000.
(60) Provisional application No. 60/141,488, filed on Jun. 29, 1999.

(51) Int. Cl. A61K 31/496; A61K 31/47; A61K 31/4709

(52) U.S. Cl. 514/253.08; 514/312

(57) ABSTRACT

This invention relates, in part, to newly identified methods of using quinolone antibiotics, particularly a gemifloxacin compound against certain pathogenic bacteria.

This invention relates, in part, to newly identified methods of using quinolone antibiotics, particularly a gemifloxacin compound against Streptococcus pneumonia bacteria, such as fluoroquinolone resistant S. pneumoniae, especially ciprofloxacin or trovafloxacin resistant S. pneumoniae.

### BACKGROUND OF THE INVENTION

Quinolones have been shown to be effective to varying degrees against a range of bacterial pathogens. However, as diseases caused by these pathogens are on the rise, there exists a need for antimicrobial compounds that are more potent than the present group of quinolones.

Gemifloxacin mesylate (SB-265805) is a novel fluoroquinolone useful as a potent antibacterial agent. Gemifloxacin compounds are described in detail in patent application PCT/KR98/00051 published as WO 98/42705. Patent application EP 688772 discloses novel quinolone(naphthyridine)carboxylic acid derivatives, including anhydrous (R,S)-7-(3-aminomethyl-4-methoxyiminopyrrolidin-1-yl)-1-cyclopropyl-6-fluoro-4-oxo-1,4-dihydro-1,8-naphthyridine-3-carboxylic acid of formula I.

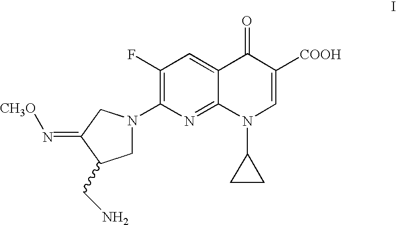

PCT/KR98/00051 discloses (R,S)-7-(3-aminomethyl-4-syn-methoxyimino-pyrrolidin-1-yl)-1-cyclopropyl-6-fluoro-4-oxo-1,4-dihydro-1,8-naphthyridine-3-carboxylic acid methanesulfonate and hydrates thereof including the sesquihydrate.

Gemifloxacin (herein “GFX”) is possesses potent gram positive activity, is active against penicillin susceptible and resistant S. pneumoniae and is also active against ciprofloxacin (herein “CFX”) and trovafloxacin (herein “TFX”) resistant isolates.

Recent surveillance in Canada indicated that the incidence of S. pneumoniae with reduced susceptibility to fluoroquinolone drugs is gradually increasing. Thus, a need existed for methods of treating these diseases. In an effort to make such methods, among other things, studies were designed to evaluate the activity of gemifloxacin against S. pneumoniae with reduced susceptibility to ciprofloxacin.

Provided herein is a significant discovery made using a gemifloxacin compound against strains Streptococcus pneumoniae, demonstrating the activity of the gemifloxacin compound used was superior to a number of quinolones as described in more detail herein. Gemifloxacin compounds are valuable compounds for the treatment of infection caused by a range of Streptococcus pneumoniae strains, including those resistant to usual oral therapy, thereby filling an unmet medical need.

### SUMMARY OF THE INVENTION

An object of the invention is a method for modulating metabolism of fluoroquinolone resistant pathogenic bacteria comprising the step of contacting fluoroquinolone resistant pathogenic bacteria with an antibacterially effective amount of a composition comprising a quinolone, particularly a gemifloxacin compound, or an antibacterially effective derivative thereof.

A further object of the invention is a method wherein said pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

Also provided by the invention is a method of treating or preventing a bacterial infection by fluoroquinolone resistant pathogenic bacteria comprising the step of administering an antibacterially effective amount of a composition comprising a quinolone, particularly a gemifloxacin compound to a mammal suspected of having or being at risk of having an infection with fluoroquinolone resistant pathogenic bacteria.

A preferred method is provided wherein said modulating metabolism is inhibiting growth of said bacteria or killing said bacteria.

A further preferred method is provided wherein said contacting said bacteria comprises the further step of introducing said composition into a mammal, particularly a human.

Further preferred methods are provided by the invention wherein said bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

The invention also provides a method for modulating metabolism of fluoroquinolone resistant pathogenic bacteria comprising the step of contacting fluoroquinolone resistant pathogenic bacteria with an antibacterially effective amount of a composition comprising a gemifloxacin compound, and a ciprofloxacin compound, or antibacterially effective derivatives thereof either compound or both compounds.

Further provided by the invention is a method of treating or preventing a bacterial infection by fluoroquinolone resistant pathogenic bacteria comprising the step of administering an antibacterially effective amount of a composition comprising a gemifloxacin compound and a ciprofloxacin compound to a mammal suspected of having or being at risk of having an infection with fluoroquinolone resistant pathogenic bacteria.

A still further method of the invention is a method for modulating metabolism of fluoroquinolone resistant pathogenic bacteria comprising the step of contacting fluoroquinolone resistant pathogenic bacteria with an antibacterially effective amount of a composition comprising a gemifloxacin compound, followed by a ciprofloxicin compound, or antibacterially effective derivatives thereof either compound or both compounds.

Another embodiment of the invention is a method of treating or preventing a bacterial infection by fluoroquinolone resistant pathogenic bacteria comprising the step of administering an antibacterially effective amount of a composition comprising a gemifloxacin compound followed by a ciprofloxicin compound to a mammal suspected of having or being at risk of having an infection with fluoroquinolone resistant pathogenic bacteria.

Various changes and modifications within the spirit and scope of the disclosed invention will become readily apparent to those skilled in the art from reading the following descriptions and from reading the other parts of the present disclosure.

### DESCRIPTION OF THE INVENTION

The present invention provides, among other things, methods for using a composition comprising a gemifloxacin compound against a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

As used herein “gemifloxacin compound(s)” means a compound having antibacterial activity described in patent application PCT/KR98/00051 published as WO 98/42705, or patent application EP 688772.

This invention was based, in part, on analyses evaluating the comparative activity of gemifloxacin against various strains of S. pneumoniae. An objective of these analyses was to determine the postantibiotic effect (herein “PAE”) of GFX in CFX and TFX susceptible and resistant S. pneumoniae.

CFX resistant, clinical isolates of S. pneumoniae were collected from across Canada. MICs were determined using a microbroth dilution technique described by the NCCLS. Topisomeriase IV (parC) and DNA gyrase (gyrA) mutations were confirmed by sequencing the QRDR region of each gene. Two fluoroquinolone susceptible and 8 resistant isolates (3 CFX resistant, 5 CFX, TFX resistant) were selected for study. The PAE was determined by exposing logarithmic phase organisms at 4× or 10×MIC for 2 hours. Antibiotics were removed using dilution into sterile media and the PAE assessed using a viable colony counting technique. The MICs of CFX ranged from 0.5 μg/ml to 64 μg/ml and the MICs of TFX ranged from 0.06 μg/ml to 8.0 μg/ml. The MICs of GFX ranged from ≦0.03 μg/ml to 0.5 μg/ml. The mean PAE of CFX in susceptible S. pneumoniae was 1.6 hrs at 4× MIC and 2.5 hrs at 10× MIC. In TFX susceptible isolates, the mean PAE at 4× MIC was 2.1 hrs and 3.2 hrs at 10× MIC. The mean PAE of GFX was 2.7 hrs at 4× MIC and 3.8 hrs at 10×MIC. There was no significant difference in the duration of the GFX PAE between CFX or TFX susceptible and resistant strains (p<0.05). In conclusion, GFX remains highly active against CFX and TFX susceptible and resistant S. pneumoniae and produces a prolonged PAE in organisms displaying diminished susceptibility to other fluoroquinolones.

In another analysis, clinical isolates of S.pneumoniae were collected across Canada and isolates having an MIC to CIP of ≧2 μg/ml were selected for further study. MICs to penicillin (herein “PEN”), CIP, levofloxacin (herein “LEV”), TFX, moxifloxacin (herein “MOX”), grepafloxacin (herein “GRE”), gatifloxacin (herein “GAT”), sparfloxacin (herein “SPA”), and gemifloxacin (herein “GFX”) were determined using a microbroth dilution technique described by the NCCLS. Topoisomerase IV (parC) and DNA gyrase (gyrA) mutations were confirmed by sequencing the QRDR region of each gene. Serotyping and PFGE were performed on all isolates. In total, 80 isolates were identified with CIP MICs≧2 μg/ml. Of these, 33 had both gyrA and parC mutations, 29 had parC mutations alone and 2 had gyrA mutations. With the exception of 7 isolates, all organisms having a CIP MIC≧8 μg/ml, had both a parC and gyrA mutation. MIC_{50/90}, are listed in Table 1. Breakpoints have not been established for all fluoroquines, thus percentage resistance was not calculated. With the exception of one cluster, serotyping and PFGE suggest that resistance is de novo and not due to clonal disemmination.

These results demonstrate that GFX, followed by MOX retain the greatest activity against S. pneumoniae with reduced susceptibility to CIP. The increasing use of fluoroquine to treat S. pneumoniae mandates continued surveillance to monitor changes in fluoroquine resistance patterns.

TABLE 1

| | | LE | | | MO | | | |
|---|---|---|---|---|---|---|---|---|
| μg/ml | CIP | V | SPA | TFX | X | GAT | GRE | GFX |
| MIC₅₀ | 8 | 2 | 0.5 | 0.5 | 0.25 | 0.5 | 0.5 | 0.06 |
| MIC₉₀ | 32 | 16 | 16 | 4 | 2 | 4 | 8 | 0.25 |
| range | 2-64 | 1-32 | .25-32 | .06-8 | .12-4 | .25-8 | .12-8 | .03-.5 |

The invention provides a method for modulating metabolism of fluoroquinolone resistant pathogenic bacteria. Skilled artisans can readily choose fluoroquinolone resistant pathogenic bacteria or patients infected with or suspected to be infected with these organisms to practice the methods of the invention. Alternatively, the bacteria useful in the methods of the invention may be those described herein.

The contacting step in any of the methods of the invention may be performed in many ways that will be readily apparent to the skilled artisan. However, it is preferred that the contacting step is a provision of a composition comprising a gemifloxacin compound to a human patient in need of such composition or directly to bacteria in culture medium or buffer.

For example, when contacting a human patient or contacting said bacteria in a human patient or in vitro, the compositions comprising a quinolone, particularly a gemifloxacin compound, preferably pharmaceutical compositions may be administered in any effective, convenient manner including, for instance, administration by topical, oral, anal, vaginal, intravenous, intraperitoneal, intramuscular, subcutaneous, intranasal or intradermal routes among others.

It is also preferred that these compositions be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a compound of the invention, a quinolone, preferably a gemifloxacin compound, and a pharmaceutically acceptable carrier or excipient. Such carriers may include, but are not limited to, saline, buffered saline, dextrose, water, glycerol, ethanol and combinations thereof. The formulation should suit the mode of administration.

Quinolone compounds, particularly gemifloxacin compounds and compositions of the methods of the invention may be employed alone or in conjunction with other compounds, such as bacterial efflux pump inhibitor compounds or antibiotic compounds, particularly non-quinolone compounds, e.g., beta-lactam antibiotic compounds.

In therapy or as a prophylactic, the active agent of a method of the invention is preferably administered to an individual as an injectable composition, for example as a sterile aqueous dispersion, preferably an isotonic one.

Alternatively, the gemifloxacin compounds or compositions in the methods of the invention may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the antibacterially effective amount is a daily dosage level of the active agent from 0.001 mg/kg to 10 mg/kg, typically around 0.1 mg/kg to 1 mg/kg, preferably about 1 mg/kg. A physician, in any event, will determine an actual dosage that is most suitable for an individual and will vary with the age, weight and response of the particular individual. The above dosages are exemplary of the average case. There can, of course, be individual instances where higher or lower dosage ranges are merited, and such are within the scope of this invention. It is preferred that the dosage is selected to modulate metabolism of the bacteria in such a way as to inhibit or stop growth of said bacteria or by killing said bacteria. The skilled artisan may identify this amount as provided herein as well as using other methods known in the art, e.g. by the application MIC tests.

A further embodiment of the invention provides for the contacting step of the methods to further comprise contacting an in-dwelling device in a patient. In-dwelling devices include, but are not limited to, surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters.

A quinolone, particularly a gemifloxacin compound or composition of the invention may be administered by injection to achieve a systemic effect against relevant bacteria, preferably a fluoroquinolone resistant pathogenic bacteria, shortly before insertion of an in-dwelling device. Treatment may be continued after surgery during the in-body time of the device. In addition, the composition could also be used to broaden perioperative cover for any surgical technique to prevent bacterial wound infections caused by or related to fluoroquinolone resistant pathogenic bacteria.

In addition to the therapy described above, a gemifloxacin compound or composition used in the methods of this invention may be used generally as a wound treatment agent to prevent adhesion of bacteria to matrix proteins, particularly fluoroquinolone resistant pathogenic bacteria, exposed in wound tissue and for prophylactic use in dental treatment as an alternative to, or in conjunction with, antibiotic prophylaxis.

Alternatively, a quinolone, particularly a gemifloxacin compound or composition of the invention may be used to bathe an indwelling device immediately before insertion. The active agent will preferably be present at a concentration of 1 μg/ml to 10 mg/ml for bathing of wounds or indwelling devices.

Also provided by the invention is a method of treating or preventing a bacterial infection by fluoroquinolone resistant pathogenic bacteria comprising the step of administering an antibacterially effective amount of a composition comprising a quinolone, particularly a gemifloxacin compound to a mammal, preferably a human, suspected of having or being at risk of having an infection with fluoroquinolone resistant pathogenic bacteria.

While a preferred object of the invention provides a method wherein said fluoroquinolone resistant pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region. Other fluoroquinolone resistant pathogenic bacteria may also be included in the methods. The skilled artisan may identify these organisms as provided herein as well as using other methods known in the art, e.g. MIC tests.

Preferred embodiments of the invention include, among other things, methods wherein said composition comprises gemifloxacin, or a pharmaceutically acceptable derivative thereof.

Each reference cited herein is hereby incorporated by reference in its entirety. Moreover, each patent application to which this application claims priority is hereby incorporated by reference in its entirety.

What is claimed is:

1. A method for modulating metabolism of fluoroquinolone resistant pathogenic bacteria comprising the step of contacting fluoroquinolone resistant pathogenic bacteria with an antibacterially effective amount of a composition comprising a gemifloxacin compound, or antibacterially effective derivatives thereof.

2. The method of claim 1 wherein said fluoroquinolone resistant pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

3. A method of treating or preventing a bacterial infection by fluoroquinolone resistant pathogenic bacteria comprising the step of administering an antibacterially effective amount of a composition comprising a gemifloxacin compound to a mammal suspected of having or being at risk of having an infection with fluoroquinolone resistant pathogenic bacteria.

4. The method of claim 3 wherein said fluoroquinolone resistant pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

5. The method of claim 1 wherein said modulating metabolism is inhibiting growth of said bacteria.

6. The method of claim 1 wherein said modulating metabolism is killing said bacteria.

7. The method of claim 1 wherein said contacting said bacteria comprises the further step of introducing said composition into a mammal.

8. The method of claim 3 wherein said mammal is a human.

9. The method of claim 7 wherein said mammal is a human.

10. The method of claim 1 wherein said bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

11. The method of claim 1 wherein said bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

12. A method for modulating metabolism of fluoroquinolone resistant pathogenic bacteria comprising the step of contacting fluoroquinolone resistant pathogenic bacteria with an antibacterially effective amount of a composition comprising a gemifloxacin compound, and a ciprofloxacin compound, or antibacterially effective derivatives thereof either compound or both compounds.

13. The method of claim 12 wherein said fluoroquinolone resistant pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

14. A method of treating or preventing a bacterial infection by fluoroquinolone resistant pathogenic bacteria comprising the step of administering an antibacterially effective amount of a composition comprising a gemifloxacin compound and a ciprofloxacin compound to a mammal suspected of having or being at risk of having an infection with fluoroquinolone resistant pathogenic bacteria.

15. The method of claim 14 wherein said fluoroquinolone resistant pathogenic bacteria is selected from the group consisting of: a ciprofloxacin resistant strain of S. pneumoniae, S. pneumoniae having a topisomerase IV (parC) mutation in the QRDR region, S. pneumoniae having a DNA gyrase (gyrA) mutation in the QRDR region, a ciprofloxacin resistant strain of S. pneumoniae having a topisomerase IV (parC) mutation in the QRDR region, a ciprofloxacin resistant strain of S. pneumoniae having a DNA gyrase (gyrA) mutation in the QRDR region, a trovafloxacin resistant strain of S. pneumoniae, a trovafloxacin resistant strain of S. pneumoniae having a topisomerase IV (parC) mutation in the QRDR region, a trovafloxacin resistant strain of S. pneumoniae having a DNA gyrase (gyrA) mutation in the QRDR region, a fluoroquinolone resistant strain of S. pneumoniae, a fluoroquinolone resistant strain of S. pneumoniae having a topisomerase IV (parC) mutation in the QRDR region, and a fluoroquinolone resistant strain of S. pneumoniae having a DNA gyrase (gyrA) mutation in the QRDR region.

17. A method for modulating metabolism of fluoroquinolone resistant pathogenic bacteria comprising the step of contacting fluoroquinolone resistant pathogenic bacteria with an antibacterially effective amount of a composition comprising a gemifloxacin compound, followed by a ciprofloxicin compound, or antibacterially effective derivatives thereof either compound or both compounds.

18. A method of treating or preventing a bacterial infection by fluoroquinolone resistant pathogenic bacteria comprising the step of administering an antibacterially effective amount of a composition comprising a gemifloxacin compound followed by a ciprofloxacin compound to a mammal suspected of having or being at risk of having an infection with fluoroquinolone resistant pathogenic bacteria.
